(19) United States
(12) Patent Application Publication
(10) Pub. No.: US 2021/0330700 A1
(43) Pub. Date: Oct. 28, 2021

(54) METHODS AND COMPOSITIONS FOR INHIBITING INFLUENZA VIRUSES USING LOW MOLECULAR WEIGHT HYDROPHOBICALLY MODIFIED POLYMERS AND POLYALKYLENE GLYCOLS

(71) Applicant: Johnson & Johnson Consumer Inc., Skillman, NJ (US)

(72) Inventors: Elizabeth Bruning, Skillman, NJ (US); Kimberly Capone, Skilllman, NJ (US); Lisa Renee Gandolfi, Skillman, NJ (US); Anthony Robert Geonnotti, III, Skillman, NJ (US); Euen Thomas Ekman-Gunn, Skillman, NJ (US); Diana Roshek Johnson, Skillman, NJ (US); Frank J. Kirchner, Skillman, NJ (US); Selina Moses, Skillman, NJ (US); Delores Santora, Skillman, NJ (US); Russel Walters, Skillman, NJ (US); Frank C. Sun, Skillman, NJ (US)

(73) Assignee: Johnson & Johnson Consumer Inc., Skillman, NJ (US)

(21) Appl. No.: 16/856,801

(22) Filed: Apr. 23, 2020

(51) Int. Cl. A61K 31/78; A61K 9/00; A61P 31/16

(52) U.S. Cl. CPC A61K 31/78; A61P 31/16; A61K 9/0014

(57) ABSTRACT

This invention relates to methods and compositions for inhibiting the transmission of influenza viruses, which entails applying a composition containing a polyalkylene glycol compound to an infectable or ingestible surface that may contain viruses. It further relates to methods and compositions for inhibiting the transmission of influenza and other enveloped viruses, which entails applying a composition containing a polyalkylene glycol compound and a low molecular weight hydrophobically-modified polymer to an infectable or ingestible surface that may contain viruses.

### FIELD OF THE INVENTION

The method of this invention relates to the use of polyalkylene glycol compounds to inhibit the transmission of influenza viruses. It also relates to compositions containing said polyalkylene glycol compounds in combination with low molecular weight hydrophobically modified polymers capable of inhibiting transmission of influenza and other enveloped viruses.

### BACKGROUND OF THE INVENTION

Infections due to enveloped viruses cause common diseases such as influenza, herpes simplex, HIV/AIDS, hepatitis B, chicken pox, shingles, small pox, and respiratory infections. While the seriousness of these diseases can range from moderately bothersome to life-threatening, these infections adversely affect the quality of life of its host and the personal, institutional and economic areas of our society. As a result, there have been substantial efforts to develop means to prevent viral infection and its spread. These efforts are complicated by viral diversity, the numerous means by which viruses are transmitted, including: direct contact, exchange of bodily fluids (e.g. saliva, sexual transmission, breast feeding), and aerosol transmission (e.g. coughing, sneezing, etc.) as well as the highly evolved measures by which viruses escape detection and/or eradication by their hosts. There have been numerous successes in the discovery and commercialization of antiviral agents administered to those who have been infected with a virus. However, these treatments often require medical prescriptions, have unwanted side effects, only work on a narrow range of viral types/strains, and/or have limited efficacy. Topically delivered antiviral treatments must also be non-irritating to the treated tissues, or risk increasing the risk of infection.

Therefore, cost effective and gentle agents with potent, broad-spectrum anti-viral activity which are capable of significantly reducing virus transmission would fill an unmet need in the antiviral armamentarium and help prevent the spread of viral infections, especially if mild properties of such agents could permit and encourage widespread, frequent usage due to superior compatibility with skin, eyes and other mucosal membranes.

Viruses have high mutation and replication rates; these properties allow rapid evolution in response to external selective pressures (i.e. drug), often leading to treatment resistance and relapse. The concern of resistance is especially salient when the antiviral compound targets a specific epitope on the virion. Due to high levels of viral genetic diversity, this narrow specificity also usually limits the range of viruses sensitive to the compound. Alternatively, other topical antiviral treatments, such as surfactants, target non-specific viral regions and are broadly effective at neutralizing diverse viruses, however, these are often irritating and toxic to human cells. Treatments that irritate tissues may result in an increased infection rate; damaging cellular membranes increases their permeability to some types of viral particles. Thus, a non-irritating yet highly effective means for eradicating viruses and significantly reducing their transmission potential would be highly desirable.

Influenza is an infectious disease caused by RNA viruses of the family Orthomyxoviridae. Influenza can be very contagious and dangerous. The disease can cause high fever, muscle pains and fatigue and can lead to serious complications, including pneumonia and fatalities. It would be desirable to be able to inhibit influenza viruses before they can infect individuals.

Most viruses (e.g., HIV and many animal viruses) have viral envelopes as their outer layer at the stage of their life-cycle when they are between host cells. Robertson et al. (March 1995). “Recombination in AIDS viruses.” Journal of Molecular Evolution. 40 (3): 249-59. Some enveloped viruses also have a protein layer called a capsid between the envelope and their genome. Id. The envelopes are typically derived from portions of the host cell membranes (phospholipids and proteins), but include some viral glycoproteins. They may help viruses avoid the host immune system. Glycoproteins on the surface of the envelope serve to identify and bind to receptor sites on the host's membrane. The viral envelope then fuses with the host's membrane, allowing the capsid and viral genome to enter and infect the host.

The cell from which the virus itself buds will often die or be weakened and shed more viral particles for an extended period. The lipid bilayer envelope of these viruses is relatively sensitive to desiccation, heat, and detergents; therefore these viruses are easier to sterilize than non-enveloped viruses, have limited survival outside host environments, and typically transfer directly from host to host. Enveloped viruses possess great adaptability and can change in a short time in order to evade the immune system. Enveloped viruses can cause persistent infections.

Classes of enveloped viruses that contain human pathogens include, e.g., DNA viruses such as Herpesvirus, Poxviruses, Hepadnaviruses, Asfarviridae; RNA viruses such as Flavivirus, Alphavirus, Togavirus, Coronavirus, Hepatitis D, Orthomyxovirus, Paramyxovirus, Rhabdovirus, Bunyavirus, Filovirus; and Retroviruses such as HIV.

### COVID-19

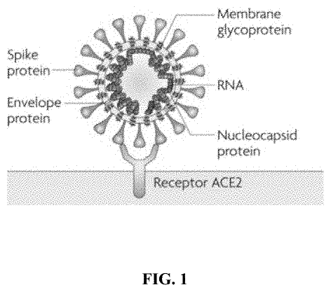

Coronaviruses (CoVs) are relatively large viruses containing a single-stranded positive-sense RNA genome encapsulated within a membrane envelope. The viral membrane is studded with glycoprotein spikes that give coronaviruses their crownlike appearance. (See FIG. 1, taken from Liu et al., Research and Development on Therapeutic Agents and Vaccines for COVID-19 and Related Human Coronavirus Diseases, ACS Cent. Sci. 2020, 6, 315-331). While coronaviruses infect both humans and animals, certain types of animals such as bats that host the largest variety of coronaviruses appear to be immune to coronavirus-induced illness. There are four classes of coronaviruses designated as alpha, beta, gamma, and delta. The betacoronavirus class includes severe acute respiratory syndrome (SARS) virus (SARS-CoV), Middle East respiratory syndrome (MERS) virus (MERS-CoV), and the COVID-19 causative agent SARS-CoV-2. Similar to SARS-CoV and MERS-CoV, SARS-CoV-2 attacks the lower respiratory system to cause viral pneumonia, but it may also affect the gastrointestinal system, heart, kidney, liver, and central nervous system leading to multiple organ failure. Current information indicates that SARSCoV-2 is more transmissible/contagious than SARS-CoV.

A number of studies have focused on elucidation of virus structure, virus transmission mechanisms/dynamics, as well as identification of antiviral agents and accurate diagnostics for virus detection. These trends reflect immense interest and desire from the scientific community, including both academic and industrial organizations as well as clinicians, to identify new methods to halt the progression of this epidemic disease and to prevent infection and transmission in the future.

COVID-19 is caused by SARS-CoV-2, a new type of coronavirus in the same genus as SARS-CoV and MERS-CoV. Viral proteins responsible for SARS-CoV-2 entry into host cells and replication are structurally similar to those associated with SARS-CoV. Thus, research and development on SARS and MERS may offer insights that would be beneficial to the development of therapeutic and preventive agents for COVID-19.

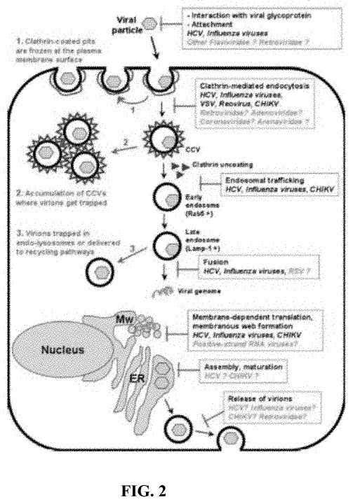

Arbidol, CAS No. 131707-23-8, which targets S protein/ACE2, is an inhibitor that may disrupt the binding of the viral envelope protein to host cells and prevent entry of the virus to the target cell has entered into clinical trials for treatment of COVID-19. See Liu et al. above and FIG. 2 below, taken from Blaising et al., Arbidol as a broad-spectrum antiviral: An update, Antiviral Research, 107 (2014) 84-94. See also Kadam et al., Structural basis of influenza virus fusion inhibition by the antiviral drug Arbidol, PNAS January 10, 2017 114 (2) 206-214.

The 2003 emergence of the severe acute respiratory disease coronavirus (SARS-CoV) demonstrated that CoVs are capable of causing outbreaks of severe infections in humans. A second severe CoV, Middle East respiratory syndrome coronavirus (MERS-CoV), emerged in 2012 in Saudi Arabia. More recently, COVID-19 identified in Wuhan, China, in December 2019, has proven particularly detrimental.

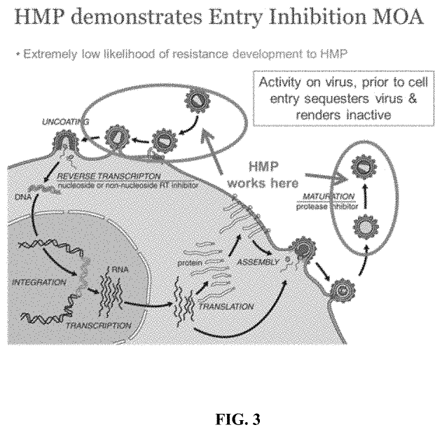

Given that the polymers of the invention have shown activity against enveloped viruses, it is expected that polymers of the invention may also show activity against COVID-19 by inhibiting entry of the virus in a host cell. See FIG. 3.

RetroVirox, San Diego, CA, has developed cell-based assays that can be used to evaluate experimental treatments against coronaviruses, including SARS-CoV-2. The Company provides testing with SARS-CoV-2 pseudoviruses to evaluate entry inhibitors against the novel coronavirus causative agent of COVID-19. The pseudovirus assay utilizes HIV pseudoviruses coated with the viral spike (S) protein of SARS-CoV-2 (Wuhan isolate). The assay, which recapitulates the mode of entry of the novel coronavirus, it can be used for, e.g., evaluate small-molecule entry inhibitors targeting the S viral protein, the ACE-2 viral receptor, or host proteases and other targets involved in SARS-CoV-2 viral entry.

U.S. Pat. Nos. 7,803,403 and 8,025,902 to Johnson & Johnson Consumer Inc. disclose personal care compositions that contain a low molecular weight, non-cross linked, linear acrylic copolymer and at least one surfactant; and a method of cleansing using said personal care compositions.

U.S. Pat. Nos. 8,343,902 and 8,329,626 to Johnson & Johnson Consumer Inc. disclose a skin cleansing composition that comprises a low molecular weight, non-crosslinked, linear acrylic copolymer and a non-ethoxylated anionic surfactant.

U.S. Pat. No. 8,329,627 to Johnson & Johnson Consumer Inc. discloses a clear skin cleansing composition that comprises a low molecular weight, non-crosslinked, linear acrylic copolymer and a blend of at least two amphoteric surfactants.

U.S. Pat. No. 8,293,845 to Lubrizol Corp. discloses a method for increasing the critical micelle concentration of a surfactant composition comprising including a linear hydrophobically modified (meth)acrylic polymer in said composition.

U.S. Pat. No. 7,892,525 to Lubrizol Advanced Materials, Inc. discloses antiperspirant compositions that comprise a cationic hydrophobically modified polymeric gelling agent and an acidic antiperspirant compound.

U.S. Pat. No. 9,068,148 to Lubrizol Advanced Materials, Inc. discloses an acrylic polymer blend that comprises at least one crosslinked acrylic copolymer and at least one acrylic linear, non-crosslinked polymer; a method for making the acrylic polymer blend; and method for thickening an aqueous composition comprising the acrylic polymer blend.

U.S. Pat. No. 9,931,290 to Lubrizol Advanced Materials, Inc. discloses a surfactant composition that comprises a surfactant and a crosslinked acrylic copolymer; and a personal care cleansing composition comprising the surfactant composition.

U.S. Pat. No. 10,517,806 to Ecolab USA Inc. claims a foaming antimicrobial dermal cleanser that comprises a cationic active ingredient; a cationic compatible surfactant; a foam boosting agent; a foam structure enhancing agent; a skin conditioning agent; and water. The reference claims a method of reducing bacterial, microbial, fungicidal, or viral population on a dermal tissue of a mammal comprising contacting the dermal tissue with the foaming antimicrobial dermal cleanser. The reference also discloses that cationic active ingredients are antimicrobial agents useful in the present invention and that the foam structure enhancing agent can be polyethyleneglycol. The reference discloses the use of S. aureus and Escherichia coli as test microbial cultures to test microbial efficacy of the formulas therein.

U.S. Pat. No. 10,435,308 to Ecolab USA, Inc. claims a composition for improving oil removal from an oil/aqueous phase solution by foam fractionation that comprises an associative thickener; a surfactant comprising a sorbitan ester; and a viscoelastic surfactant, wherein the viscoelastic surfactant is a betaine, amine oxide, and/or ethoxylated fatty amine. The reference discloses that the composition may be used in, e.g., cleaning agents, cosmetics, pickles, aqueous pigment pastes, automotive finishes, industrial coatings, printing inks, lubricating greases, plaster paints and wall paints, textile coatings, pharmaceutical preparations, crop protection formulations, filler dispersions, adhesives, detergents, wax dispersions, polishes, auxiliaries for tertiary mineral oil production etc.

U.S. Published Application No. 20160262999 to Ecolab USA, Inc. claims an antimicrobial dermal concentrate that comprises a cationic active ingredient; a foam boosting surfactant; a foam boosting copolymer; a foam stabilizing structure; and water. The reference claims that the concentrate can be used to reduce bacterial, microbial, fungicidal or viral population on a dermal tissue of a mammal. The reference discloses that cationic active” is the ingredient that provides antimicrobial activity. The reference discloses that the concentrate may contain a skin conditioner such as polyethylene glycol.

Menachery et al., Pathogenic Influenza Viruses and Coronaviruses Utilize Similar and Contrasting Approaches To Control Interferon-Stimulated Gene Responses, American Society of Microbiology, 2014, 5(3): 1-11, discloses that influenza viruses and coronaviruses exhibit differences in terms of replication, immune stimulation, and overall lethality.

Li, Structure, Function and Evolution of Coronavirus Spike Proteins, Annu. Rev. Virul. 2016, 3(1):237-261, discusses the evolution of two critical functions of coronavirus spike proteins, receptor recognition and membrane fusion, in the context of the corresponding functions from other viruses and host cells.

The cited references are incorporated by reference in their entirety herein.

Neutrogena Corp, Los Angeles, California, markets and sells a Neutrogena® Ultra Gentle Daily Cleanser product that contains the use of potassium acrylates copolymer as a viscosity increasing agent.

Johnson & Johnson Consumer Inc. markets and sells products, including Johnson's Head to Toe Baby Wash; Johnson's Baby Moisture Wash; and Johnson's Baby Wipes that contain the use of potassium acrylates copolymer as a viscosity increasing agent.

Hand sanitizers are generally used to decrease infectious agents on the hands. They are available as liquids, gels, and foams. Alcohol-based versions and non-alcohol based versions are available. Alcohol-based versions typically contain some combination of isopropyl alcohol, ethanol (ethyl alcohol), or n-propanol, with versions containing 60% to 95% alcohol being the most effective. Care should be taken as they are flammable. Alcohol-based hand sanitizer works against a wide variety of migroorganisms. Non-alcohol based versions, which typically contain benzalkonium chloride or triclosan, are less effective than alcohol-based ones.

In 2020, BlueWillow Biologics, Inc. launched NanoBio Project nasal antiseptic solution containing OTC monograph benzalkonium chloride. The product is applied by thoroughly swabbing the skin inside of each nostril.

### SUMMARY OF THE INVENTION

This invention relates to a method of inhibiting entry of influenza viruses into cells comprising, consisting essentially of and consisting of contacting said viruses with an anti-viral composition comprising, consisting essentially of and consisting of at least one polyalkylene glycol compound in an amount effective to inhibit entry of these viruses into cells. It further relates to compositions and methods of inhibiting influenza and other enveloped viruses comprising, consisting essentially of and consisting contacting influenza and other enveloped viruses with compositions comprising, consisting essentially of and consisting of at least one polyalkylene glycol compound and at least one low molecular weight hydrophobically modified polymer.

Surprisingly, we have found that compositions containing polyalkylene glycol compounds are effective in inhibiting influenza viruses, although they are not generally effective against other enveloped viruses.

Surprisingly, we have also found that low concentrations of certain low molecular weight hydrophobically modified polymers known for their gentle properties are able successfully to inhibit entry of enveloped viruses into host cells and thus inhibit transmission of viruses to the hosts. We believe that these polymers would not encounter or engender some of the historical problems with antiviral treatments, such as drug resistance, narrow breadth of neutralization and host cellular toxicity. The low molecular weight hydrophobically modified polymers useful in the methods and compositions of this invention are broadly active against several viral types and across multiple viral strains. Additionally, these polymers work through a non-specific mechanism of entry inhibition, thereby increasing their chances for inhibitory success and decreasing the likelihood of resistance. Furthermore, as these polymers are exceptionally gentle on mucosal tissues, they have little or no toxicity to human tissues.

Our bodies are challenged by viruses on a daily basis and our immune system, including our skin barrier, is designed to minimize the number of viruses that reach infectable surfaces. The low molecular weight hydrophobically modified polymers useful in the methods and compositions of this invention block the ability of the virus to bind to and/or enter cells, thereby reducing the probability that an infectious virus can reach a target cell and cause a systemic infection. Viral infection is partially the result of a stochastic process—the more viruses that come in contact with infectable cells, the more likely that tissue is to be infected—therefore, use of these polymers to block infectious viruses benefits the immune system, further reduces chances of infection and promotes general good health.

The methods and compositions of this invention using low molecular weight hydrophobically modified polymers are surprisingly effective at reducing the number of infectious virions across a broad range of viral types and strains, while remaining gentle and non-irritating to human tissues. We would expect that compositions containing other low molecular weight hydrophobically modified polymers and polyalkylene glycol compounds would be similarly gentle and non-irritating to human and other living organism tissues.

### DETAILED DESCRIPTION OF THE PREFERRED EMBODIMENTS

As used herein, the term “infectable surface” means a surface of a living animal the cells of which may be infected by a virus, including mammals such as human beings. Examples of such infectable surfaces are external skin tissues and mucosal tissues. Mucosal tissues include oral, ocular, nasal, vaginal and rectal tissue.

As used herein, the term “ingestible surface” refers to the surface of foods, including the surface of fruits and vegetables. As used herein, the term “hard surface” refers to surfaces found in the environment such as tables, chairs, walls, and other inanimate surfaces with which skin and/or mucosal tissue may come into contact and on which viruses may reside. The term “internal surface” refers to internal organ surfaces and internal tissues and fluids within the body of a living organism.

As used herein, the term “virus” means a small infectious agent that can replicate only inside living cells or organisms. Virus particles contain the following parts: genetic material made from either RNA or DNA and a protein coat that protects the genetic material. In some cases, virus particles are surrounded by an envelope of lipids around the protein coat when the virus particles are outside a cell. Virus particles that contain such an envelope of lipids are referred to herein as “enveloped viruses”. Enveloped viruses include the following organisms: influenza viruses, poxviridae including, but not limited to, molloscum contagiosum, chickenpox, smallpox and other pox viruses, Herpesviridae including herpes simplex virus 1 and herpes simplex virus 2, retroviridae including Lentivirus including Human Immunodeficiency Virus.

As used herein, the term “surfactant” is a surface active agent, or a substance that, when dissolved in water or an aqueous solution, reduces its surface tension or the interfacial tension between it and another liquid.

As used herein, the term “inhibiting transmission” means one or more of the following: (i) impeding the entry of a virus into a host cell; (ii) substantially stopping the introduction of a virus from one individual, infectable surface or contact surface to another; and/or (iii) reducing damage to mucosal membranes such that the membranes retain their integrity and protect against infection by the virus.

As used herein, the hydrophilic-lipophilic balance (“HLB”) is a measure of the degree to which a surfactant is hydrophilic or lipophilic, as determined by calculating values for different regions of the surfactant molecule in accordance with methods known to those of skill in the art.

Preferably, the method of this invention relates to a method of inhibiting entry of influenza viruses into cells comprising, consisting essentially of and consisting of contacting said viruses with an anti-viral composition comprising, consisting essentially of and consisting of at least one polyalkylene glycol compound. The methods of this invention also relate to a method of inhibiting influenza and other enveloped viruses comprising, consisting essentially of and consisting of contacting said viruses with an anti-viral composition comprising, consisting essentially of and consisting of at least one polyalkylene glycol compound and at least one low molecular weight hydrophobically modified polymer in an amount effective to inhibit entry of viruses into cells. The methods of this invention further include the application of the compositions set forth herein onto infectable surfaces as well as onto ingestible surfaces. The methods further include contacting viruses with the anti-viral compositions of this invention.

We have found that, surprisingly, compositions containing polyalkylene glycols are effective in inhibiting transmission of influenza viruses, although they are not effective in inhibiting other enveloped viruses. We theorize that compositions containing both polyalkylene glycol compounds and low molecular weight hydrophobically modified polymers would be effective in inhibiting transmission of enveloped viruses across a broad spectrum of enveloped virus species.

The methods of this invention also include the application of the compositions of this invention to ingestible surfaces such as food as well as to hard surfaces into which skin and mucosal tissue might come into contact. As such, the presence of the compositions of this invention would work to inhibit entry of viruses present on ingestible and hard surfaces into cells contained on skin and mucosa.

Preferably, the compositions of this invention contain at least about 55% water. Most preferably, the compositions of this invention are substantially free of surfactant having an HLB greater than about 12. Notwithstanding the foregoing, the compositions of this invention may additionally contain surfactants having an HLB of less than 12. However, the compositions of this invention may also optionally contain from about 0.375% to about 9% of surfactant having an HLB greater than 12. However, the surfactant levels of the compositions of this invention should be sufficiently low so as not to produce irritation of the skin of a mammal upon exposure or tissue disruption to the cells of the skin or mucosa of said mammal. Such tissue disruption results in providing easier viral entry into the cells. Nonetheless, compositions capable of being used for cleansing as well as virus inhibition are desirable for application to living organisms, including mammals and preferably, humans.

Preferably, the compositions of this invention contain at least one polyalkylene glycol compound. Such compounds are polyether compounds that are polymers containing alkylene glycol monomeric units.

More preferably, the polyalkylene glycol compounds useful in the compositions of this invention are polyethylene glycols and/or polypropylene glycols. Most preferably, high molecular weight polyethylene glycols are present, said molecular weight preferably ranging from about 200 to about 2,000,000. More preferably, the range of molecular weights should be from about 200 Da to about 2,000,000 Da, and most preferably the range should be from about 6000 Da to about 200,000 Da. Such preferable compounds include, but are not limited to polyethylene glycol 6000 (molecular weight of 6000). The polyalkylene glycol compounds should preferably be present in the compositions of this invention in amounts sufficient to inhibit influenza viruses. Preferably, they should be present in amounts of from about 0.05% to about 10% by weight of the composition, more preferably from about 0.1% to about 9% and most preferably from about 0.5% to about 3%.

The compositions of this invention may be applied to infectable surfaces of a living entity including mammals, reptiles, birds, fish, bacteria, and the like. Infectable surfaces of these living entities may include, but are not limited to, skin and mucosal tissues. Mucosal tissue includes, but is not limited to oral tissue, nasal tissue, vaginal tissue, rectal tissue or a combination thereof. Importantly, the compositions and methods of this invention do not disrupt these biological surfaces or cause significant irritation of those surfaces.

### Polymeric Material

Examples of polymeric materials useful in the compositions and methods of this invention include low-molecular weight acrylic, polysaccharide, cellulose, starch polymers, other ethylenically-unsaturated polymers, polyesters, polycarbonates, polyanhydrides, polyamides, polyurethanes, polyureas, polyimides, polysulfones, polysulfides, combinations of two or more thereof, and the like. Examples of suitable low molecular weight acrylic polymers include hydrophobically-modified acrylic, polysaccharide, cellulose, starch polymers, combinations of two or more thereof, and the like. Suitable low molecular weight acrylic polymers include hydrophobically-modified acrylic polymers, as well as other acrylic polymers, any of which may be formed via solution, suspension, precipitation, dispersion, emulsion, inverse emulsion, microemulsion, micellar polymerization methods, and combinations of two or more thereof. The acrylic polymers for use in the present invention may be derived from any one or more monomers selected from the group consisting of (meth)acrylates, (meth)acrylamides, vinyl ethers, esters, and amides, allyl ethers, esters, amines, and amides, itaconates, crotonates, styrenics, and olefins. The acrylic polymers may be nonionic hydrophilic, nonionic hydrophobic, anionic, cationic, zwitterionic, nonassociative macromer, associative macromer, or multifunctional/crosslinking.

As used herein the term “low molecular weight” polymer refers to a polymer having a number average molecular weight (Mₙ) of about 100,000 or less as measured by gel permeation chromatography (GPC) calibrated with a poly(methyl methacrylate) (PMMA) standard. In certain preferred embodiments, low-molecular weight polymers are those having molecular weight ranges of from about 5,000 to about 80,000 Mₙ, more preferably from about 10,000 to about 50,000 Mₙ, and more preferably between about 15,000 and 40,000 Mₙ.

Certain hydrophobically-modified polymers and methods of making such polymers are described in U.S. Pat. No. 6,433,061, issued to Marchant et al. and incorporated herein by reference. The polymeric materials useful in the composition of this invention are preferably non-crosslinked, linear acrylic copolymers that are very mild to the skin and mucosa. These non-crosslinked, linear polymers are preferably of low molecular weight having a number average molecular weight of 100,000 or less as measured by gel permeation chromatography (GPC) calibrated with a poly(methyl methacrylate) (PMMA) standard (as used herein, unless otherwise specified, all number average molecular weights (Mₙ) refer to molecular weight measured in such manner). Thus, the polymeric material functions as a copolymeric compound. The copolymeric compound is polymerized from at least two monomeric components. The first monomeric component is selected from one or more α,β-ethylenically unsaturated monomers containing at least one carboxylic acid group. This acid group can be derived from monoacids or diacids, anhydrides of dicarboxylic acids, monoesters of diacids, and salts thereof. The second monomeric component is hydrophobically modified (relative to the first monomeric component) and is selected from one or more α,β-ethylenically unsaturated non-acid monomers containing a C₁ to C₉ alkyl group, including linear and branched C₁ to C₉ alkyl esters of (meth)acrylic acid, vinyl esters of linear and branched C₁ to C₁₀ carboxylic acids, and mixtures thereof. In one aspect of the invention the second monomeric component is represented by the formula:

CH₂═CRX

wherein R is hydrogen or methyl; X is —C(O)OR¹ or —OC(O)R²; R¹ is linear or branched C₁ to C₉ alkyl; and R² is hydrogen or linear or branched C₁ to C₉ alkyl. In another aspect of the invention R¹ and R² is linear or branched C₁ to C₈ alkyl and in a further aspect R¹ and R² are linear or branched C₂ to C₅ alkyl.

Thus, preferably the hydrophobically modified polymers useful in the compositions and methods of this invention comprise, consist essentially of and consist of a low molecular weight, non-crosslinked, linear acrylic copolymer derived from at least one first monomeric component selected from the group consisting of (meth)acrylic acid and at least one second monomeric component selected from the group consisting of one or more C₁ to C₉ alkyl (meth)acrylates, wherein the low molecular weight copolymer has a number average molecular weight of about 100,000 or less.

Exemplary first monomeric components include (meth)acrylic acid, itaconic acid, citraconic acid, maleic acid, fumaric acid, crotonic acid, aconitic acid, and mixtures thereof. Exemplary second monomeric components include ethyl (meth)acrylate, butyl (meth)acrylate, 2-ethylhexyl (meth)acrylate, vinyl formate, vinyl acetate, 1-methylvinyl acetate, vinyl propionate, vinyl butyrate, vinyl 2-ethylhexanoate, vinyl pivalate, vinyl neodecanoate, and mixtures thereof. As used herein, the terms “(meth)acrylic” acid and “(meth)acrylate” are meant to include the corresponding methyl derivatives of acrylic acid and the corresponding alkyl acrylate For example, “(meth)acrylic” acid refers to acrylic acid and/or methacrylic acid and “(meth)acrylate” refers to alkyl acrylate and/or alkyl methacrylate.

More preferably, said first monomeric component is selected from the group consisting of (meth)acrylic acid and said second monomeric component is selected from the group consisting of at least one C₁ to C₉ alkyl (meth)acrylate.

The non-crosslinked, linear acrylic copolymer compounds useful in the compositions and methods of this invention can be synthesized via free radical polymerization techniques known in the art. In one aspect of the invention, the amount of the first monomeric component to the second monomeric component utilized ranges from about 20:80 wt. % to about 50:50 wt. %, based on the total weight of all of the monomers in the polymerization medium. In another aspect the weight ratio of the first monomeric component to the second monomeric component is about 35:65 wt. %, and in a further aspect the weight ratio of first monomeric component to second monomeric component is about 25:75 wt. %, all based on the total weight of all monomers in the polymerization medium.

Methods of synthesizing the polymers useful in the compositions and methods of this invention may be found in U.S. Pat. No. 6,433,061 which is hereby incorporated herein by reference.

The linear copolymeric materials useful in the methods and compositions of this invention preferably have a viscosity of 500 mPa·s or less (Brookfield RVT, 20 rpm, spindle no. 1) at a 5 wt. % polymer solids concentration in deionized water and neutralized to pH 7 with an 18 wt. % NaOH solution. The viscosity can range from about 1 to about 500 mPa·s in another aspect, from about 10 to about 250 mPa·s in a further aspect, and from about 15 to about 150 mPa·s in a still further aspect.

Preferably, the low molecular weight, non-crosslinked linear acrylic copolymer present in the compositions and methods of this invention is potassium acrylates copolymer.

The low molecular weight hydrophobically modified polymers useful in the compositions and methods of this invention are preferably present in said compositions in amounts that are effective to inhibit substantially the entry of enveloped viruses into cells and/or to inhibit virus transmission to cells. Accordingly, the compositions and methods of this invention inhibit virus entry into said cells and results in the reduction of the potential for viral infection. Preferably, they should be present in the compositions of this invention in an amount of from about 0.00005% to about 10% percent by weight of the composition. Even more preferably, they should be present in the amount of from about 0.00005% to about 3% by weight of the composition. More preferably, the low molecular weight hydrophobically modified polymers are present in an amount of from about 0.00005% to about 0.5 percent by weight of the composition. Most preferably, the low molecular weight hydrophobically modified polymers are present in an amount of from about 0.00005% to about 0.01% percent by weight of the composition.

While influenza viruses are enveloped, we have found that compositions containing low molecular weight hydrophobically modified polymers without the presence of one or more polyalkylene glycols do not inhibit the transmission of such viruses. Surprisingly, we have found that compositions containing polyalkylene glycol compounds do act to inhibit influenza viruses. Thus, we theorize that compositions containing both classes of compounds would act to inhibit influenza as well as other enveloped viruses.

The composition of this invention may be in the form of a lotion or liquid capable of being applied on the surface of the skin or on an inanimate surface that can contain viruses or bacteria. It may also be a composition which is applied to a mucosal surface such as the surfaces of the nasal cavity or vaginal cavity and can be used as a vaginal microbicide. These types of composition may be more viscous and may be based on a gel formation. The compositions of this invention may be coated onto an absorbent article such as a vaginal or nasal tampon for placement in contact with mucosal surfaces to inhibit viruses in such biologic environments. The compositions of this invention may also be formulated in such a delivery form that they may be injected into the body at appropriate sites where viruses may reside on internal surfaces.

The compositions of this invention may be made into a wide variety of product types that include but are not limited to liquids, lotions, creams, gels, sticks, sprays, shaving creams, ointments, cleansing liquid washes and solid bars, shampoos, pastes, powders, mousses, wipes, patches, wound dressing and adhesive bandages, hydrogels and films. These product types may contain several types of cosmetically acceptable topical carriers including, but not limited to solutions, emulsions (e.g., microemulsions and nanoemulsions), gels, solids and liposomes. The following are non-limiting examples of such carriers. Other carriers may be formulated by those skilled in the art of formulating such product types.

Preferred compositions of the invention include polymer containing gels; polymer containing drops, including, e.g., eye drops; polymer containing contact lens solutions; polymer containing sprays, e.g., face/body sprays, nasal sprays, and mouth and throat sprays; and polymer containing inhalants.

The compositions of the invention may also be used as a coating on or in personal protective equipment. Personal protective equipment, which is commonly referred to as “PPE”, is any equipment worn to minimize exposure to a variety of hazards. Examples of PPE include full body suits, gloves, gowns, masks, respirators and eye and foot protection.

The topical compositions useful in the methods of this invention may be formulated as solutions. Solutions preferably contain an aqueous solvent (e.g., from about 50% to about 99.99% or from about 90% to about 99% of a cosmetically acceptable aqueous solvent).

Topical compositions useful in the methods of this invention may be formulated as a solution containing an emollient. Such compositions preferably contain from about 2% to about 50% of an emollient(s). As used herein, “emollients” refer to materials used for the prevention or relief of dryness, as well as for the protection of the skin. A wide variety of suitable emollients is known and may be used herein. Sagarin, Cosmetics, Science and Technology, 2nd Edition, Vol. 1, pp. 32-43 (1972) and the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7^{th} Edition, 1997) (hereinafter “ICI Handbook”) contain numerous examples of materials for use in the compositions and methods of this invention.

A lotion may also be made from such a solution. Lotions preferably contain from about 1% to about 20% (more preferably, from about 5% to about 10%) of an emollient(s) and from about 50% to about 90% (more preferably, from about 60% to about 80%) of water.

Another type of product that may be formulated from a solution is a cream. A cream preferably contains from about 5% to about 50% (more preferably, from about 10% to about 20%) of an emollient(s) and from about 45% to about 85% (more preferably from about 50% to about 75%) of water.

Yet another type of product that may be formulated from a solution is an ointment. An ointment may contain a simple base of animal or vegetable oils or semi-solid hydrocarbons. An ointment may preferably contain from about 2% to about 10% of an emollient(s) plus from about 0.1% to about 2% of a thickening agent(s). A more complete disclosure of thickening agents or viscosity increasing agents useful herein may be found in Sagarin, Cosmetics, Science and Technology, 2nd Edition, Vol. 1, pp. 72-73 (1972) and the ICI Handbook pp. 1693-1697.

The topical compositions useful in the methods of this invention may also be formulated as emulsions. If the carrier is an emulsion, preferably from about 1% to about 10% (e.g., from about 2% to about 5%) of the carrier contains an emulsifier(s). Emulsifiers may be nonionic, anionic or cationic. Suitable emulsifiers are set forth in, for example, U.S. Pat. Nos. 3,755,560, 4,421,769, McCutcheon's Detergents and Emulsifiers, North American Edition, pp. 317-324 (1986), and the ICI Handbook, pp.1673-1686, which are incorporated herein by reference.

The compositions of this invention may be wash-off cleansing composition preferably containing a low level of surfactant as set forth above, including anionic, cationic, amphoteric, or nonionic surfactants. Such surfactants contained in the compositions of this invention include those set forth in copending U.S. patent applications Ser. No. 12/822,329 (filed Jun. 24, 2010), Ser. No. 12/976,573 (filed Dec. 22, 2010) and Ser. No. 13/166,445 (filed Jun. 22, 2011). Surfactants that are preferable for use in the compositions of this invention include, but are not limited to: anionic types, including: alkyl carboxylates; alkyl sulfonates; alkyl ether sulfonates; alkylbenzenesulfonates; naphthalenesulfonates; olefin sulfonates; alkyl sulfates; alkyl sulfonates; sulfated natural oils & fats; sulfated esters; sulfated alkanolamides; alkylphenols, ethoxylated & sulfated; nonionic types, including ethoxylated aliphatic alcohol; polyoxyethylene surfactants; carboxylic esters of fatty acid ; alkyl polyethylene glycol esters; glycol esters of fatty acids; carboxylic amides; monoalkanolamine condensates; polyoxyethylene fatty acid amides; cationic types, including dodecyl trimethyl ammonium chloride (15), cetyl ethyl morpholinium ethosulphate(25-30), polyethoxylated c12 amine (15 mol ethylene oxide), polyethoxylated c18 amine (15 mol ethylene oxide); quaternary ammonium salts; amines with amide linkages; polyoxyethylene alkyl & alicyclic amines; n,n,n′,n′ tetrakis substituted ethylenediamines; 2-alkyl 1-hydroxethyl 2-imidazolines, amphoteric types, including n-alkyl 3-aminopropionic acid/sodium salt; n-alkyl 3-iminodipropionate, disodium salt; n-carboxymethyl n dimethyl n-9 octadecenyl ammonium hydroxide; n-cocoamidethyl n hydroxyethylglycine, sodium salt, alkyl amidopropyl betaine salts, alkyl ampho acetate salts and the like.

Preferably, surfactants useful in the compositions and methods of this invention may be selected from but not limited to, the following: Potassium cetyl phosphate, hydrogenated palm glycerides (available from Symrise AG of Branchburg, N.J.), polysorbate 20, 60 and 80 (available from UNIQEMA of Bridgewater, N.J.), 2-methyloxirane and oxirane (available from BASF Corporation of Florham Park, N.J.) and the like.

Lotions and creams may also be formulated as emulsions. Preferably such lotions contain from 0.5% to about 5% of an emulsifier(s). Such creams would preferably contain from about 1% to about 20% (more preferably, from about 5% to about 10%) of an emollient(s); from about 20% to about 80% (more preferably, from 30% to about 70%) of water; and from about 1% to about 10% (more preferably, from about 2% to about 5%) of an emulsifier(s).

Other compositions useful in the methods of this invention include gels and liquid compositions that may be applicable to mucosal surfaces for inhibiting viral transmission.

Mucosal surfaces include but are not limited to the vagina, rectum, nasal passages, mouth and throat. Preferably, such compositions should include at least one polyhydric alcohol, including glycerin, polyethylene glycol, propylene glycol, sorbitol or a combination thereof. Other polyhydric alcohols know to those of ordinary skill in the art may be used in the compositions and methods of this invention, including polyethylene glycols ranging from molecular weight of from about 300 to about 1450. Preferably, there should be from about 0.1 to about 50% by weight of glycerin and from about 2 to about 40% by weight of propylene glycol.

The mucosal compositions of this invention should also contain one or more water-soluble cellulose-derived polymers. Preferably, such polymers should be a cellulose gum such as one or more hydroxyalkylcellulose polymer. More preferably, the hydroxyalkylcellulose polymer should be one or more of hydroxyethylcellulose, hydroxymethylcellulose, hydroxypropylcellulose, hydroxypropylmethylcellulose and the like. Preferably, the cellulose-derived polymer should be present in the compositions of this invention in the amount of from about 0.1 to about 2% by weight of the composition.

The compositions of this invention may be prepared in accordance with those methods and processes known to those of skill in the art, or in accordance with the methods of preparation of this invention. For example, water-soluble components such as glycerin, propylene glycol, sorbitol, inorganic base, preservatives, and the like may be dissolved in water and to that combination cellulose-derived polymers may be added. Another method of preparation is mixing all the ingredients into a slurry without water, and then adding the slurry to water.

The composition is preferably substantially free of surfactant, including anionic, cationic, amphoteric, or nonionic surfactants.

Included in a liquid or lotion formation of the composition may be water, oils, preservatives, emulsifiers, viscosity enhancers, emollients, electrolytes, fragrance, buffers, pH modifiers, skin protectants, metal ion sequestrants and the like.

The compositions of this invention may be useful in formulating hand and/or body washes, fruit and/or vegetable washes, ingestible compositions, suppositories, nasal sprays, post-surgical tampons and the like, which may be applied to surfaces or placed in the body to inhibit transmission of viruses. The compositions of this invention may be coated onto an absorbent article such as a vaginal or nasal tampon for placement in contact with mucosal surfaces to inhibit viruses in such biologic environments.

### Methods

There are various testing methods that have been employed herein to evaluate different aspects of the methods and compositions of this invention and their effects upon skin, mucosa and viruses when exposed to the compositions of the invention.

### Evaluation of Activity Against Influenza A:

Inhibition of virus-induced cytopathic effects (CPE) and cell viability following influenza virus replication in MDCK cells was measured by XTT tetrazolium dye. MDCK cells cultured in DMEM supplemented with 10% FBS, 2 mM L-glutamine, 100 U/ml penicillin, 100 μg/ml streptomycin 1 mM sodium pyruvate, and 0.1 mM NEAA are seeded in a 96-well flat-bottomed plate at a cell density of 1×10⁴ cells per well in a volume of 100 μL. The plates are incubated at 37° C./5% CO₂ for 24 hours. Following the incubation, media was removed from the cell monolayers and the cells were washed with DPBS. Compounds were diluted serially ½ logarithmically and 6 concentration of each was added to the cells in triplicate. The influenza A virus strain A/PR/8/34 was diluted to a pre-determined titer and added to the plate and the cultures were incubated at 37° C15% CO2 for four days at which time cell viability was measured by XTT staining. The optical density of the cell culture plate will be determined spectrophotometrically at 450 and 650 nm using Softmax Pro 4.6 software. Percent CPE reduction of the virus-infected wells and the percent cell viability of uninfected drug control wells were calculated using 4-parameter curve fitting. Ribavirin was evaluated in parallel as a positive control. Following the incubation, the polymers are serially diluted in half logarithmic increments (6 concentrations total) and 100 μL of each concentration is added to the cells in triplicate.

### Materials:

A low molecular weight hydrophobically modified polymer, Potassium Acrylates Copolymer (Lubrizol, Brecksville, Ohio) was used in the compositions of this invention as the low molecular weight hydrophobically modified polymer.

### EXAMPLE 1

Inventive examples E1-E6 and Comparative Examples C1-C2: Preparation of Compositions to be Tested

The compositions of E1-E6, D1 and C1-C2 were prepared according to the descriptions set forth below with materials in the amounts listed in Table 1. Compositions E1-E6, D1 and C1-C2 are in accordance with the compositions and methods of this invention. Compositions C1-C2 are comparative compositions.

TABLE 1

| Ingredient | D1 | E2 | E1 | E3 | E4 | E5 | E6 | | |
|---|---|---|---|---|---|---|---|---|---|
| INCI name | w/w % | w/w % | w/w % | w/w % | w/w % | w/w % | w/w % | C1 | C2 |
| Potassium Acrylates | 0.09 | — | | | | | 0.009 | 0.09 | 0.9 |
| Copolymer | | | | | | | | | |
| PEG200 | — | | 0.5 | | | | | | |
| PEG6000 | — | 0.5 | | | | | 0.5 | 0.5 | 0.5 |
| Cocamidopropyl | — | — | | | | | | | |
| Betaine | | | | | | | | | |
| PEG100,000 (Polyox | | | | 0.5 | | | | | |
| WSR-N-10 PEG-2M) | | | | | | | | | |
| PEG200,000 Polyox | | | | | 0.5 | | | | |
| WSR-N-80 PEG-5M) | | | | | | | | | |
| PEG2,000,000 Polyox | | | | | | 0.5 | | | |
| WSR-N-60k PEG-45M) | | | | | | | | | |
| Sodium laureth Sulfate | — | — | | | | | | | |
| PEG 80 Sorbitan | — | — | | | | | | | |
| Laurate | | | | | | | | | |

*expressed in % w/w actives
Each of the compositions of Table 1 was independently prepared as follows:
D1- 0.3 g of Potassium Acrylates Copolymer (Activity 30%) was mixed with 99.7 g of deionized water with slight heating at 65 C. and the pH adjusted to 7.08 using 20% Sodium Hydroxide solution.
E1- 0.5 g of PEG200 was dissolved in 99.5 g of deionized water with slight heating to 65 C. and the pH adjusted to 6.5 using 20% Sodium Hydroxide solution.
E2- 0.5 g of PEG6000 was dissolved in 99.5 g of deionized water with slight heating to 65 C. and the pH adjusted to 6.3 using 20% Sodium Hydroxide solution.
E3- 0.5 g of PEG100,000 (Polyox WSR-N-10 PEG-2M was dissolved in 99.5 g of deionized water with slight heating to 65 C. and the pH adjusted to 6.41 using 20% Citric Acid solution.
E4- 0.5 g of PEG200,000 (Polyox WSR-N-80 PEG-5M was dissolved in 99.5 g of deionized water with slight heating to 65 C. and the pH adjusted to 6.69 using 20% Citric Acid solution.
E5- 0.5 g of PEG2,000,000 (Polyox WSR-N-60k PEG-45M was dissolved in 99.5 g of deionized water with slight heating to 65 C. and the pH adjusted to 6.45 using 20% Citric Acid solution.
E6 0.5 g of PEG6000 was combined with 0.04 g of Potassium Acrylates Copolymer (Activity 30%) in 99.5 g of deionized water with slight heating to 65 C. and the pH adjusted to 6.52 using 20% Sodium Hydroxide solution.
C1 0.5 g of PEG6000 was combined with 0.3 g of Potassium Acrylates Copolymer (Activity 30%) in 99.2 g of deionized water with slight heating to 65 C. and the pH adjusted to 7.09 using 20% Sodium Hydroxide solution.
C2 0.5 g of PEG6000 was combined with 3.01 g of Potassium Acrylates Copolymer (Activity 30%) in 96.5 g of deionized water with slight heating to 65 C. and the pH adjusted to 6.57 using 20% Sodium Hydroxide solution.

### EXAMPLE 3

Activity of Embodiments against Influenza

Following the protocol described above, embodiments E1-E6, D1 and C1-C2 were tested against Influenza A/PR/8/34 (Table 3).

TABLE 3

| | Influenza A/PR/8/34 | Influenza A/PR/8/34 |
|---|---|---|
| Embodiment | EC50 (μg/ml) | TC50 (μg/ml) |
| D1 | >1500 | 71.1 |
| E1 | 2256 | >2500 |
| E2 | 1605 | >2500 |
| E3 | 2009 | >2500 |
| E4 | 811 | >2500 |
| E5 | 970 | >2500 |
| E6 | 2167 | >2500 |
| C1 | >2500 | >2500 |
| C2 | >2500 | >2500 |

Note:
all EC50 and TC50 concentrations for E1-E6, C1-C2 are the concentration of the PEG in the sample. D1 shows the concentration of the potassium acrylates copolymer (activity 30%) in the sample.

What is claimed is:

1. A method of inhibiting entry of influenza viruses into cells comprising contacting said viruses with an anti-viral composition comprising at least one polyalkylene glycol compound having a molecular weight in an amount effective to inhibit entry of viruses into cells.

2. A method of inhibiting entry of influenza and other enveloped viruses into cells comprising contacting said viruses with an anti-viral composition comprising at least one polyalkylene glycol compound having a molecular weight from in an amount effective to inhibit entry of viruses into cells and at least one low molecular weight hydrophobically-modified polymer.

3. A method according to claim 1 further comprising applying said anti-viral composition to infectable surfaces of a subject.

4. A method according to claim 1 wherein said infectable surfaces comprise one or more of the group consisting of skin and mucosal tissue of a subject.

5. A method according to claim 3 wherein said mucosal tissue comprises tissue selected from the group consisting of oral tissue, ocular tissue, nasal tissue, vaginal tissue, or rectal tissue and a combination thereof.

6. A method according to claim 1 wherein said low molecular weight hydrophobically modified polymer is selected from the group consisting of low-molecular weight acrylic, polysaccharide, cellulose, starch polymers, other ethylenically-unsaturated polymers, polyesters, polycarbonates, polyanhydrides, polyamides, polyurethanes, polyureas, polyimides, polysulfones, polysulfides, combinations of two or more thereof, and the like.

7. A method according to claim 6 wherein said low molecular weight hydrophobically modified polymer comprises a polymer derived from at least one first monomeric component selected from the group consisting of (meth)acrylic acid and at least one second monomeric component selected from the group consisting of one or more C₁ to C₉ alkyl (meth)acrylates, wherein the low molecular weight copolymer has a number average molecular weight of about 100,000 or less.

8. A method according to claim 1 wherein said low molecular weight hydrophobically modified polymer is present in said composition in an amount of from about 0.00005% to about 3% percent by weight of the composition.

9. A method according to claim 1 wherein said composition further comprises at least 55% of water.

10. A method according to claim 9 wherein said composition comprises at least 97% of water.

11. A method according to claim 2 wherein said viruses are selected from the group consisting of influenza, poxviridae, herpesviridae, retroviridae Lentivirus and a combination thereof.

12. A method according to claim 1 wherein said polyalkylene glycol is selected from the group consisting of polyethylene glycol and polypropylene glycol and a mixture thereof.

13. A method according to claim 12 wherein said polyalkylene glycol is polyethylene glycol having a molecular weight from about 200 to about 2,000,000.

14. A method according to claim 13 wherein said polyalkylene glycol is polyethylene glycol having a molecular weight from about 6,000 to about 200,000.

15. A method according to claim 1 wherein said inhibition of influenza virus entry into said cells results in the reduction of potential for viral infection.

16. A method according to claim 1 wherein the anti-viral composition does not substantially disrupt biological surfaces.

17. A method of inactivating enveloped viruses comprising contacting infectable surfaces with an anti-viral composition comprising at least one low molecular weight hydrophobically modified polymers in an amount effective to inhibit entry of viruses into cells, wherein said composition is substantially free of surfactant.

18. A method of inhibiting entry of influenza viruses according to claim 18 further comprising contacting said viruses with said anti-viral composition.

19. An anti-viral composition comprising at least one low molecular weight hydrophobically modified polymers and at least one polyalkylene glycol in an amount effective to inhibit entry of viruses into cells and at least 55% water, wherein said composition is substantially free of surfactant.

20. An anti-viral composition comprising at least one low molecular weight hydrophobically modified polymers and at least one polyalkylene glycol in an amount effective to inhibit entry of viruses into cells and at least 55% water, wherein said composition is substantially free of surfactant having an HLB greater than 12.

21. A method of inhibiting the transmission of influenza and other enveloped viruses comprising applying to non-biological surfaces a composition comprising at least one low molecular weight hydrophobically modified polymers and at least one polyalkylene glycol in an amount effective to inhibit entry of influenza viruses into cells wherein said composition is substantially free of surfactant.

22. A method of inhibiting the transmission of influenza viruses comprising applying to ingestable surfaces a composition comprising at least one low molecular weight hydrophobically modified polymers and at least one polyalkylene glycol in an amount effective to inhibit entry of viruses into cells wherein said composition is substantially free of surfactant.

23. A composition according to claim 20 wherein said composition comprises a dosage form selected from the group consisting of: a liquid, a lotion, a cream, a gel, a stick, a spray, a shaving cream, an ointment, a cleansing liquid wash, a solid bar, a shampoo, a paste, a powder, a mousse, a wipe, a patch, a wound dressing, an adhesive bandage, a hydrogel and a film.

24. A composition according to claim 20 wherein said hydrophobically modified low molecular weight polymer comprises a low molecular weight, non-crosslinked, linear acrylic copolymer derived from at least one first monomeric component selected from the group consisting of (meth)acrylic acid and at least one second monomeric component selected from the group consisting of one or more C₁ to C₉ alkyl (meth)acrylates, wherein the low molecular weight copolymer has a number average molecular weight of about 100,000 or less.

25. A composition according to claim 24 wherein said hydrophobically modified low molecular weight polymer is potassium acrylates copolymer.
